(19) United States
(12) Patent Application Publication
(10) Pub. No.: US 2015/0051298 A1
(43) Pub. Date: Feb. 19, 2015

(54) METHODS FOR PRODUCING OPTIMAL STABLE NANOEMULSIONS AND FORMULATIONS OBTAINED THEREFROM

(71) Applicants: David J. McClements, Northampton, MA (US); Amir H. Saberi, Amherst, MA (US); Yuhua Chang, Amherst, MA (US); Lynne McLandsborough, Sunderland, MA (US)

(72) Inventors: David J. McClements, Northampton, MA (US); Amir H. Saberi, Amherst, MA (US); Yuhua Chang, Amherst, MA (US); Lynne McLandsborough, Sunderland, MA (US)

(21) Appl. No.: 14/458,517

(22) Filed: Aug. 13, 2014

Related U.S. Application Data
(60) Provisional application No. 61/865,778, filed on Aug. 14, 2013.

(51) Int. Cl. A61K 31/015; A23L 1/30; A01N 25/04; A61K 47/12; A01N 31/08

(52) U.S. Cl. CPC A61K 31/015; A61K 47/12; A01N 31/08; A01N 25/04; A23L 1/3008; A23L 1/3002
USPC 514/731; 514/763

(57) ABSTRACT

A method for producing stable nanoemulsions having a desired droplet size and functional properties tailored for use in a specific application (which is referred to as substantially optimizing composition) that includes selecting an aqueous phase; the aqueous phase comprising at least one ingredient from water, surfactant, co-surfactant and co-solvent, the aqueous phase being selected such that a stable nanoemulsions having a desired droplet size and functional properties tailored for use in a specific application is obtained, and selecting an organic phase comprising at least two ingredients from lipophilic component, oil, surfactant, co-surfactant and cosolvent, the organic phase being selected such that a stable nanoemulsions having a desired droplet size and functional properties tailored for use in a specific application is obtained, a nanoemulsion being formed when the organic phase is mixed with the aqueous phase.

### CROSS-REFERENCE TO RELATED APPLICATIONS

This application claims the benefit of priority to U.S. Provisional Application No. 61/865,778, entitled METHODS FOR PRODUCING OPTIMAL STABLE NANOEMULSIONS AND FORMULATIONS OBTAINED THEREFROM, filed on Aug. 14, 2013, the entire contents of which are incorporated herein by reference for all purposes.

### STATEMENT REGARDING FEDERALLY SPONSORED RESEARCH OR DEVELOPMENT

This invention was made partially with U.S. Government support from the US Department of Agriculture (USDA) under USDA Grant #: 2011-67021-30160. The Government may have certain rights in the invention

### BACKGROUND

These teachings relate generally to methods for producing substantially optimal stable nanoemulsions and to formulations obtained therefrom.

Colloidal delivery systems are needed in the food, supplements, cosmetics, personal care, and pharmaceutical industries to encapsulate functional lipophilic components so that they can be dispersed within aqueous media. These functional lipophilic components may be vitamins, nutraceuticals, triacylglycerol oils (e.g. polyunsaturated fats, conjugated linoleic acid (CLA), flavors, colors, antimicrobials, preservatives, etc. Functional lipophilic components include a variety of different kinds of molecules with different functional attributes, such as triacylglycerols (clouding agents, carrier oils, nutrients, and bioactive lipids), citrus oils (flavoring agents), essential oils (antimicrobials), phytosterols (nutraceuticals), carotenoids (colorants, antioxidants, and nutraceuticals), oil-soluble vitamins (essential nutrients), and lipophilic drugs (pharmaceuticals). These lipophilic components vary in their molecular and physicochemical properties, such as polarities, surface activities, densities, viscosities, melting points, and boiling points. Consequently, different colloidal delivery systems are often needed for different kinds of lipophilic components and for different types of food or pharmaceutical matrices.

Essential oils are natural compounds produced by aromatic plants as secondary metabolites that have antioxidant, antiradical, and antimicrobial properties, and therefore they have been widely used as functional ingredients in food, cosmetic, and pharmaceutical applications. The major constituents in commercial essential oils can be classified into three classes: phenols; terpenes; and, aldehydes. Some essential oils have been shown to exert strong antibacterial activities against food-borne pathogens, leading to their broad application as natural antimicrobial additives to extend the shelf life of food and beverage products. The fact that essential oils are considered to be “natural” components makes them highly desirable for use in many commercial applications due to growing consumer demand for natural rather than synthetic additives. However, the utilization of essential oils is often limited owing to their relatively low water-solubility. A simple way to solve this problem is to encapsulate essential oils in oil-in-water (O/W) emulsions or nanoemulsions. These emulsion-based delivery systems have previously been used to encapsulate various kinds of lipophilic bioactive components, including antitumor agents, anti-inflammatory agents, vitamins, and antimicrobials. After encapsulation, the lipophilic components can be easily incorporated into aqueous-based foods and beverages due to their improved water-dispersibility.

The term “Vitamin E” is used to describe a group of lipophilic molecules that have related molecular structures and biological properties, including the α, β, γ, and ε derivatives of tocopherol and tocotrienol. Epidemiological and experimental studies have shown that α-tocopherol may play an important role in prevention of chronic disease and carcinogenesis. However, the utilization of α-tocopherol as a functional ingredient in food and beverages is currently limited owing to its heat and oxygen sensitivity, poor water-solubility, and low and variable bioavailability. A number of different α-tocopherol delivery systems have been investigated as potential means to overcome these problems. Among them, emulsion-based systems have been identified as being particularly suitable for the oral delivery of oil-soluble vitamins. Emulsion-based systems can be fabricated using relatively simple processing operations and commercially viable ingredients.

Three of the most widely used colloidal delivery systems consist of small lipid particles dispersed within an aqueous phase are microemulsions, nanoemulsions, and, emulsions. The main differences between these three colloidal systems are their thermodynamic stability and particle dimensions. Microemulsions are thermodynamically stable dispersions of oil, water and surfactant (and possibly cosurfactants) that typically contain lipid particles with radii less than 100 nm. Nanoemulsions (r<100 nm) and emulsions (r>100 nm) are both thermodynamically unstable dispersions that are distinguished according to their droplet dimensions. There are certain advantages and disadvantages to the commercial utilization of each of these colloidal delivery systems. Microemulsions and nanoemulsions contain small particles that only scatter light weakly and so they tend to be optically clear or only slightly turbid. They also tend to have good stability to gravitational separation and particle aggregation once they have been successfully formulated due to their small particle size. The small size of the particles may also increase the bioactivity of any encapsulated components. On the other hand, these systems often require relatively large amounts of surfactant to formulate them. Since microemulsion formation is thermodynamically driven, their existence is governed by intrinsic and extrinsic thermodynamic variables such as temperature, pressure, concentration, etc. Microemulsions may therefore become unstable if these variables change during processing, packaging, transport, storage or at the point of sale. Emulsions contain relatively large droplets that scatter light strongly and so they tend to be optically opaque or highly turbid, which is desirable for some applications and undesirable for others. They are also usually less stable to gravitational separation and particle aggregation than microemulsions and nanoemulsions. On the other hand, the amount of surfactant needed to form stable emulsions is usually considerably less than that required to form microemulsions and nanoemulsions.

Nanoemulsions (NEs) have similar compositions, structures, and thermodynamic properties as the conventional emulsions that are currently widely used as emulsion-based delivery systems in many commercial applications. However, these two types of colloidal dispersion can be distinguished from each other based on the size of the droplets they contain. Nanoemulsions are categorized as having a mean droplet radius less than 100 nm (i.e., diameter<200 nm), whereas conventional emulsions are categorized as containing larger droplets. These differences in droplet dimensions can have a pronounced influence on the functional properties of emulsion-based delivery systems. Nanoemulsions can be produced with droplet dimensions appreciably smaller than the wavelength of light (d<<λ) so that they do not scatter light strongly, thereby making them either transparent or only slightly turbid. These systems can be used to incorporate lipophilic bioactive compounds into transparent aqueous based products, such as some functional foods and beverages, sauces and syrups. The small size of the droplets in nanoemulsions may also improve their stability to gravitational separation, flocculation, and coalescence when compared to conventional emulsions. It has been suggested that the bioavailability of encapsulated lipophilic compounds increases as the droplet size in emulsions decreases, although this is likely to be highly system dependent. The activity of encapsulated compounds may increase as the droplet size in emulsions decreases.

In general, nanoemulsions can be prepared using two main approaches: high-energy and low-energy. High-energy approaches utilize mechanical devices that produce intense disruptive forces that intermingle and breakup the oil and water phases, such as high pressure homogenizers, microfluidizers, and ultrasound generators. In contrast, low-energy approaches mainly rely on the internal chemical energy of the system to form small droplets. As a consequence, low energy approaches are usually much more energy efficient and require simpler equipment than high energy approaches, and they are often more effective at producing small droplet sizes. A number of different low-energy approaches have been developed to form NEs, including spontaneous emulsification (SE), phase inversion temperature (PIT), phase inversion composition (PIC), and emulsion inversion point (EPI) methods. These methods are not widely used in the food industry at present, and where they are used there is still a relatively poor understanding of the factors affecting their performance. Low energy approaches may have advantages over high-energy approaches for certain applications: they are often more effective at producing very fine droplets; they have lower equipment and energy costs; they are simpler to implement. On the other hand, there are also some potential disadvantages of low-energy systems, including limitations on the types of oils and surfactants that can be used to form stable nanoemulsions, and the fact that relatively high surfactant-to-oil ratios (SOR) are typically needed to produce them.

There are many commercial products that should be optically transparent or only slightly turbid, and therefore any colloidal delivery system should contain very small droplets (r<60 nm) that do not scatter light strongly. FIG. 1 shows the appearance of nanoemulsions (r=30 nm) and conventional emulsions (r=150 nm) with similar compositions (20 wt % tetradecane, 6% surfactant). The small droplets in nanoemulsions scatter light weakly thereby appearing clear or slightly cloudy.

In view of the number of different applications, there is a need for a method for preparing stable nanoemulsions with desired functional performance.

### BRIEF SUMMARY

In one embodiment of the method of these teachings, the method of these teachings for producing stable nanoemulsions having a desired droplet size and functional properties tailored for use in a specific application (which is referred to as substantially optimizing composition) includes selecting an aqueous phase; the aqueous phase comprising at least one ingredient from water, surfactant, co-surfactant and co-solvent, the aqueous phase being selected such that a stable nanoemulsions having a desired droplet size and functional properties tailored for use in a specific application is obtained, and selecting an organic phase comprising at least two ingredients from lipophilic component, oil, surfactant, co-surfactant and cosolvent, the organic phase being selected such that a stable nanoemulsions having a desired droplet size and functional properties tailored for use in a specific application is obtained, a nanoemulsion being formed when the organic phase is mixed with the aqueous phase.

The spontaneous emulsification method is particularly suitable for utilization within the food and beverage industry because of its simplicity and low-cost. Practically, this method involves titrating an organic phase into an aqueous phase with constant stirring. FIG. 2 shows that nanoemulsions can be formed spontaneously when an organic phase is mixed with an aqueous phase. The movement of a water-dispersible substance (e.g., surfactant) from the organic phase to the aqueous phase leads to spontaneous droplet formation. The organic phase may consist of a number of different components, including: the functional lipophilic component to be encapsulated; one or more carrier oils; one or more surfactants/cosurfactants; one or more solvents/cosolvents. The aqueous phase may also consist of a number of different components: including water; one or more cosolvents; one or more surfactants/cosurfactants; buffering agents; salts.

The composition of the cosolvent and aqueous phases must be carefully controlled to achieve three major goals: (1) initial formation of ultrafine droplets; (2) long-term stability of the ultrafine droplets (e.g., against flocculation, coalescence, partial coalescence, or Ostwald ripening); (3) functional performance of encapsulated bioactive component (such as bioavailability, antimicrobial activity, chemical stability, optical clarity, textural properties, and flavor profile). A schematic diagram showing the rationale used by the optimization scheme is shown in FIGS. 3a and 3b. FIG. 3a shows a Schematic diagram showing that the organic phase and aqueous phase compositions must be carefully controlled in order to produce nanoemulsions containing small droplets that are stable, and have the appropriate functional performance. FIG. 3b shows a Schematic diagram showing that the organic phase and aqueous phase compositions must be selected to produce: (i) small droplets; (ii) stable droplets; (iii) optimum functional performance. Substantially Optimum conditions must be matched for all three parameters. There is usually only a highly limited range of organic and aqueous phase compositions where all three criteria can be obtained (FIG. 3b). The traditional methods of optimizing nanoemulsions involve trial-and-error experiments or statistical methodologies (such as surface response methodology), and usually focus one or two of the above criteria.

In these teachings, a method for preparing stable nanoemulsions with desired functional performance by substantially optimizing the aqueous phase and oil phase compositions in order to identify the parameter range that satisfies all three criteria (particle size, stability and desired functional properties) is presented. Substantially optimizing is performed by obtaining from experiments the variation of the three criteria and selecting the common range.

The precise mechanism for the formation of ultrafine droplets is currently unknown, but it is believed that when the organic phase is titrated into the aqueous phase any water-dispersible substances within it (such as surfactants or cosolvents) move from the organic phase into the aqueous phase. As a result, the interfacial region at the boundary separating the organic and aqueous phases breaks up leading to the spontaneous formation of ultrafine oil droplets that move into the aqueous phase (FIG. 2).

Surprisingly, it has been found, in these teachings, that stable nanoemulsions containing ultrafine oil droplets can be produced that also maintain their good functional performance by carefully controlling the aqueous phase and organic phase compositions simultaneously, as well as the preparation conditions. Usually, in conventional situations, it is not possible to optimize initial particle size and long-term stability and functional performance in a particular system using the spontaneous emulsification method.

The formulation of these teachings having a desired droplet size and functional properties tailored for use in a specific application includes an aqueous phase, the aqueous phase including at least one ingredient from water, surfactant, co-surfactant and co-solvent, type and concentration of the aqueous phase being selected such that a stable nanoemulsion having a desired droplet size and functional properties tailored for use in the predetermined application is obtained, and an organic phase including at least two ingredients from lipophilic component, oil, surfactant, co-surfactant and cosolvent, type and concentration of the organic phase being selected such that a stable nanoemulsion having a desired droplet size and functional properties tailored for use in the predetermined application is obtained, a nanoemulsion being formed when the organic phase is mixed with the aqueous phase.

In one instance, the formulation of these teachings having a desired droplet size and functional properties tailored for use in a specific application includes an oil phase comprising at least two oils blended together at different mass ratios; the at least two oils and the mass ratios being selected such that a stable nanoemulsion having a desired droplet size and functional properties tailored for use in a specific application is obtained, an aqueous phase comprising at least two polar components blended together at different mass ratios; the at least two polar components and the mass ratios being selected such that a stable nanoemulsions having a desired droplet size and functional properties tailored for use in a specific application is obtained, at least two surfactants blended at different mass ratios; the at least two surfactants and the mass ratios being selected such that a stable nanoemulsions having a desired droplet size and functional properties tailored for use in a specific application is obtained, the at least two surfactants and the oil phase being blended together at different mass ratios; wherein the mass ratios for blending the at least two surfactants with the oil phase are selected such that a stable nanoemulsion is obtained, a nanoemulsion being formed when the organic phase is mixed with the aqueous phase.

For a better understanding of the present teachings, together with other and further needs thereof, reference is made to the accompanying drawings and detailed description and its scope will be pointed out in the appended claims.

### BRIEF DESCRIPTION OF THE DRAWINGS

FIG. 1 shows the appearance of nanoemulsions (r=30 nm) and conventional emulsions (r=150 nm) with similar compositions (20 wt % tetradecane, 6% surfactant);

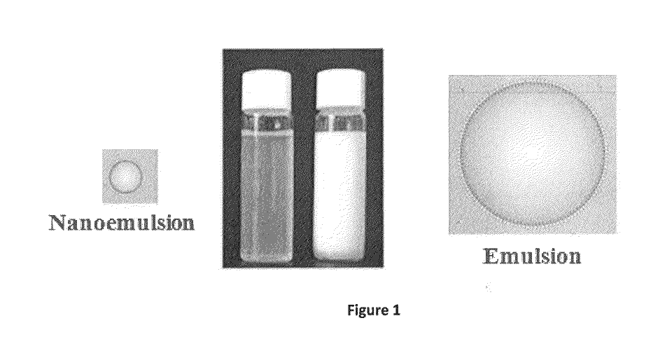

FIG. 2 shows that nanoemulsions can be formed spontaneously when an organic phase is mixed with an aqueous phase;

FIG. 3a shows a schematic diagram showing that the organic phase and aqueous phase compositions must be carefully controlled in order to produce nanoemulsions containing small droplets that are stable, and have the appropriate functional performance;

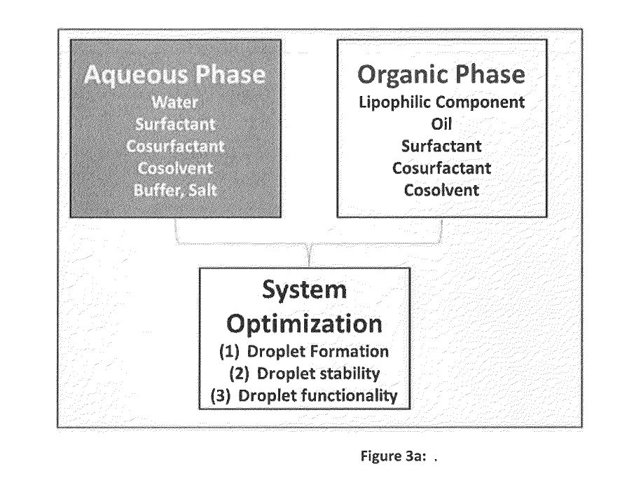

FIG. 3b shows a schematic diagram showing that the organic phase and aqueous phase compositions must be selected to produce: (i) small droplets; (ii) stable droplets; (iii) optimum functional performance;

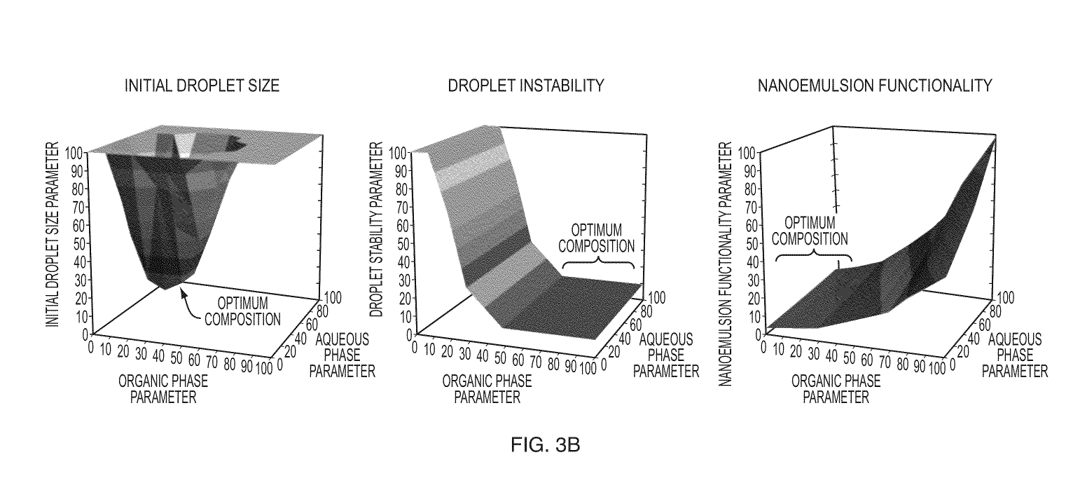

FIG. 4 represents the effect of cosolvent (glycerol) concentration on the initial mean particle diameter and polydispersity index of nanoemulsions produced by titrating an organic phase (fish oil, lemon oil, and Tween 80) into an aqueous phase (water and glycerol) using the spontaneous emulsification method. For this oil phase, the smallest size was found at a particular glycerol concentration;

FIG. 5 shows the effect of ethanol concentration on increase in mean particle diameter after 24 hours storage of nanoemulsions produced by titrating an organic phase (fish oil, lemon oil, and Tween 80) into an aqueous phase (water and glycerol) using the spontaneous emulsification method; For this organic phase, the droplets were highly unstable to growth at high ethanol concentrations;

FIG. 6 Shows the effect of lemon oil concentration on mean particle diameter of nanoemulsions produced using oil (fish oil and lemon oil), surfactant (Tween 80) and aqueous phase (ethanol and water) after 1 and 24 hours storage;

FIG. 7 Shows the effect of corn oil concentration on mean particle diameter of nanoemulsions produced using oil (tributyrin and corn oil), surfactant (β-lactoglobulin) and aqueous phase (water) after 1 and 24 hours storage; Rapid droplet growth occurs in samples with low corn oil concentration;

FIG. 8 Shows the effect of oil phase composition (% corn oil in oil phase) on the bioaccessibility of encapsulated β-carotene in nanoemulsions produced using oil (lemon oil and corn oil), surfactant (sucrose monopalmitate) and aqueous phase (water); The bioaccessibility decreases as the concentration of corn oil decreases;

FIG. 9 Shows the effect of oil phase composition (% wt of carvacrol in oil phase) on mean particle diameter of emulsions and nanoemulsions produced by spontaneous emulsification. Emulsions and nanoemulsions were prepared using 10 wt % oil (Carvacrol+MCT), 10 wt % surfactant (TWEEN® 80) and 80 wt % water (pH 3.5 citrate buffer solution) at a stirring speed of 500 rpm at ambient temperature (≈25° C.);

FIG. 10 shows the Dependence of the minimal inhibitory concentration (MIC) of carvacrol in nanoemulsions with varying carvacrol concentration in the lipid phase; The determination of MICs were performed in a nutrient MEB medium (pH 3.5) against four acidic resistant yeasts as indicated; Please note that the MIC values equaling to 10000 ppm (as shown in the figure) actually means>10000 ppm;

FIG. 11 shows the Effect of surfactant type on mean particle diameter and physical stability of emulsions produced by spontaneous emulsification. Oil phase=2.5% Carvacrol+7.5% MCT; Surfactant was 10 wt % in system;

FIG. 12 shows the Effect of surfactant concentration on mean particle diameter and physical stability of emulsions produced by spontaneous emulsification. Surfactant (Tween 80) concentration in emulsion varied from 2.5 to 20 wt % as indicated. Oil phase=2.5% Carvacrol+7.5% MCT;

FIG. 13 shows the Effect of oil phase composition (% wt of orange oil in oil phase) on mean particle diameter of emulsions and nanoemulsions produced by spontaneous emulsification; Emulsions and nanoemulsions were prepared using 10 wt % oil (orange oil+MCT), 10 wt % surfactant (TWEEN® 80) and 80 wt % water (pH 3.5 citrate buffer solution) at a stirring speed of 500 rpm at ambient temperature (≈25° C.). From left to right, the photograph shows orange oil concentrations in the lipid phase of 0, 10, 15, 20, 25, 30, 40, 50, 60, 80, 100 wt %, respectively;

FIG. 14 shows the Effect of surfactant concentration on mean particle diameter and physical stability of emulsions produced by spontaneous emulsification; Surfactant (Tween 80) concentration in emulsion varied from 2.5 to 20 wt % as indicated. Oil phase=4% orange oil+6% MCT;

FIG. 15 shows the Effect of surfactant type on mean particle diameter and physical stability of emulsions produced by spontaneous emulsification; Oil phase=4% orange oil+6% MCT. Surfactant was 20 wt % in system; and

FIG. 16 shows the Effect of surfactant type and oil phase composition on mean particle diameter of emulsions produced by spontaneous emulsification. Oil phase composition (varying ration of orange oil+MCT, total oil=10 wt %) was indicated as in the legend. Surfactant was 20 wt % in system; the results indicate that Tween 40 can load a larger amount of orange oil (6%) producing transparent nanoemulsions with very small particle sizes (d=25 nm), while Tween 60 can load the smallest amount of orange oil (4%), and Tween 80 in the middle (5% orange oil); Other types of surfactants cannot produce transparent orange oil nanoemulsions, no matter of the oil phase.

### DETAILED DESCRIPTION

The following detailed description presents the currently contemplated modes of carrying out the invention. The description is not to be taken in a limiting sense, but is made merely for the purpose of illustrating the general principles of the invention, since the scope of the invention is best defined by the appended claims.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.”

“Substantially optimizing,” as used herein, refers to determining a range of input parameters around an optimum value for each of a number of required criteria and selecting a common range of the input parameters over which the number of required criteria are close to or equal to optimum. In vernacular terms, such a common range is referred to as “a sweet spot.”

In one embodiment of the method of these teachings, the method of these teachings for producing stable nanoemulsions having a desired droplet size and functional properties tailored for use in a specific application (which is referred to as substantially optimizing composition) includes selecting an aqueous phase; the aqueous phase comprising at least one ingredient from water, surfactant, co-surfactant and co-solvent, the aqueous phase being selected such that a stable nanoemulsion having a desired droplet size and functional properties tailored for use in a specific application is obtained, and selecting an organic phase comprising at least two ingredients from lipophilic component, oil, surfactant, co-surfactant and cosolvent, the organic phase being selected such that a stable nanoemulsions having a desired droplet size and functional properties tailored for use in a specific application is obtained, a nanoemulsion being formed when the organic phase is mixed with the aqueous phase.

To prepare stable nanoemulsions with an appropriate functional performance using the spontaneous emulsification method it has been shown, in these teachings, that the organic and aqueous phase compositions must be carefully controlled. Hereinbelow, three important design parameters that must be taken into account when substantially optimizing these compositions are highlighted:

(1). Initial Droplet Size

Initially, the composition of the organic and aqueous phases must be optimized to ensure that small droplets are produced in the nanoemulsions. This can be achieved in a number of ways, such as (but not limited to):

Organic phase: The type and concentrations of functional lipophilic components, carrier oils, cosolvents, surfactants, cosurfactants can be varied.

Aqueous phase: The type and concentrations of water, cosolvents, surfactant, cosurfactant, buffer, salt, pH can be varied.

(2). Droplet Stability

Once small droplets have been produced it is important to ensure that they remain stable during manufacturer, storage, transport, handling, and utilization. This can be achieved in a number of ways, such as (but not limited to):

Organic phase: The type and concentrations of functional lipophilic components, carrier oils, cosolvents, surfactants, cosurfactants can be varied.

Aqueous phase: The type and concentrations of water, cosolvents, surfactant, cosurfactant, buffer, salt, pH can be varied.

Storage conditions: Storage temperature, light exposure, and mechanical stresses can also be controlled to ensure stability.

(3). Nanoemulsion Functional Performance

Once stable nanoemulsions have been formed it is important to ensure that they maintain their desired functional performance, such as antimicrobial activity, bioaccessibility, chemical stability, flavor profile, etc. This can be achieved in a number of ways, such as (but not limited to):

Organic phase: The type and concentrations of functional lipophilic components, carrier oils, cosolvents, surfactants, cosurfactants, antioxidants etc. can be varied.

Aqueous phase: The type and concentrations of water, cosolvents, surfactant, cosurfactant, buffer, salt, pH, antimicrobials, antioxidants etc. can be varied.

One detailed embodiment of these teachings is presented hereinbelow:

(1). In some situations a single component oil phase may be used to form a stable nanoemulsion. However, in many cases it is necessary to blend two or more different types of oils together to optimize nanoemulsion formation, stability, and performance. The optimum oil phase composition is established by blending the oils together at different mass ratios. For example, for a two phase system the composition may be varied from 0 to 100% for Oil 1 and from 100 to 0% for Oil 2. The types of oils selected will depend on the required particle size, stability, and functional performance of the nanoemulsion. For example, one oil may facilitate the formation of ultrafine particles, whereas the other oil may facilitate good long term stability (e.g., a ripening inhibitor) or functional performance (e.g., an antimicrobial).

(2). In some situations a single aqueous phase may be used to form a stable nanoemulsion (e.g., pure water). However, in many cases it is necessary to blend two or more different types of polar components, such as water, cosolvents, polyols, sugars, etc. The optimum aqueous phase composition is established by blending the polar components together at different mass ratios. For example, for a two phase system the composition may be varied from 0 to 100% for Polar Component 1 and from 100 to 0% for Polar Component 2. The types of polar component selected depend on the required particle size, stability, and functional performance of the nanoemulsion.

(3). In some situations a single surfactant may be used to form a stable nanoemulsion. However, in many cases it is necessary to blend two or more different types of surfactant together. The optimum surfactant composition is established by blending the surfactants together at different mass ratios. For example, for a two phase surfactant system the composition may be varied from 0 to 100% for Surfactant 1 and from 100 to 0% for Surfactant 2. The types of surfactant selected depend on the required particle size, stability, and functional performance of the nanoemulsion.

(4). The ratio of Surfactant (S) to Oil (O) used in the spontaneous emulsification method has to be optimized to form a stable nanoemulsion. The optimum surfactant-to-oil ratio (SOR) is established by blending the surfactant and oil phase together at different mass ratios. For example, the composition may be varied from 5 to 95% for Surfactant and from 95 to 5% for Oil.

(5). The final concentration of surfactant and oil in the surfactant (S), oil (O) and water (W) mixture formed after formation of the nanoemulsion by spontaneous emulsification must also be optimized. In the total SOW system, the final surfactant content will typically vary from <0.1 to 60%, the oil content from <0.1 to 60%, and the water content from 10 to >99%. (With S+O+W=100%). Optimization results in a formulation of these teachings that includes an oil, a surfactant and water, a mass ratio of the oil, a mass ratio of the surfactant and a mass ratio of the water being selected such that nanoemulsions having a desired droplet size and functional properties tailored for use in the predetermined application are obtained.

(6). After formation, it may be necessary to rapidly dilute the nanoemulsion formed by spontaneous formation to increase its long-term stability. This can sometimes be achieved by diluting the nanoemulsion with water or by using an aqueous solution that contains additional stabilizing components (such as surfactants, minerals, buffers, proteins, polysaccharides, antioxidants, etc.). Typically, dilution factors ranging from 1 to 100 are sufficient to improve the stability of the nanoemulsions.

The formulation of these teachings having a desired droplet size and functional properties tailored for use in a specific application includes an aqueous phase, the aqueous phase including at least one ingredient from water, surfactant, co-surfactant and co-solvent, type and concentration of the aqueous phase being selected such that a stable nanoemulsion having a desired droplet size and functional properties tailored for use in the predetermined application is obtained, and an organic phase including at least two ingredients from lipophilic component, oil, surfactant, co-surfactant and cosolvent, type and concentration of the organic phase being selected such that a stable nanoemulsion having a desired droplet size and functional properties tailored for use in the predetermined application is obtained, a nanoemulsion being formed when the organic phase is mixed with the aqueous phase.

In one embodiment, the aqueous phase in the formulation of these teachings also includes buffers and salts.

In another embodiment, the organic phase in the formulation of these teachings includes an oil phase comprising of these two oils blended together at different mass rations; the at least two oils and the different mass rations being selected such that a stable nanoemulsion having a desired droplet size and functional properties tailored for specific use in a predetermined application is obtained.

In one instance, the organic phase in the formulation of these teachings also includes at least two surfactants blended at other different mass rations; the at least two surfactants and the other different mass rations being selected such that a stable nanoemulsion having a desired droplet size and functional properties tailored for specific use in a predetermined application is obtained.

In another instance, the organic phase in the formulation of these teachings includes surfactant and oil and a ratio of surfactant to oil is selected such that a stable nanoemulsion having a desired droplet size and functional properties tailored for use in the predetermined application is obtained

In yet another embodiment, the organic phase in the formulation of these teachings includes surfactant and oil, the aqueous phase includes water, and a concentration of surfactant and oil in a surfactant, oil and water mixture is selected such that a stable nanoemulsion having a desired droplet size and functional properties tailored for use in the predetermined application is obtained.

In a further embodiment, the aqueous phase in the formulation of these teachings includes at least two polar components blended together a different mass ratios; the at least two components and the different mass ratios being selected such that a stable nanoemulsion having a desired droplet size and functional properties tailored for specific use in a predetermined application is obtained.

In one instance, the formulation of these teachings also includes another aqueous solution comprising stabilizing components. The stabilizing components, in one instance, are selected from at least one of surfactants, minerals, buffers, proteins, polysaccharides and antioxidants.

In another instance, the formulation of these teachings having a desired droplet size and functional properties tailored for use in a specific application includes an oil phase comprising at least two oils blended together at different mass ratios; the at least two oils and the mass ratios being selected such that a stable nanoemulsions having a desired droplet size and functional properties tailored for use in a specific application is obtained, an aqueous phase comprising at least two polar components blended together at different mass ratios; the at least two polar components and the mass ratios being selected such that a stable nanoemulsions having a desired droplet size and functional properties tailored for use in a specific application is obtained, at least two surfactants blended at different mass ratios; the at least two surfactants and the mass ratios being selected such that a stable nanoemulsions having a desired droplet size and functional properties tailored for use in a specific application is obtained, the at least two surfactants and the oil phase being blended together at different mass ratios; wherein the mass ratios for blending the at least two surfactants with the oil phase are selected such that a stable nanoemulsion is obtained, a nanoemulsion being formed when the organic phase is mixed with the aqueous phase. The formulation would be obtained as detailed above.

It has been found, in these teachings, that stable ultrafine droplets can be produced by controlling the compositions of the aqueous phase and/or organic phase used to prepare nanoemulsions by the spontaneous emulsification method. To produce stable nanoemulsions using this method it is important to simultaneously control a number of different compositional and preparation parameters.

### Exemplification

In order to further illustrate these teachings, exemplary embodiments are presented hereinbelow. It should be noted that these teachings are not limited only to these exemplary embodiments. In some exemplary embodiments one or a number of different factors that must be accounted for are presented using a variety of functional lipophilic components as exemplary embodiments.

2.1. Aqueous Phase Composition

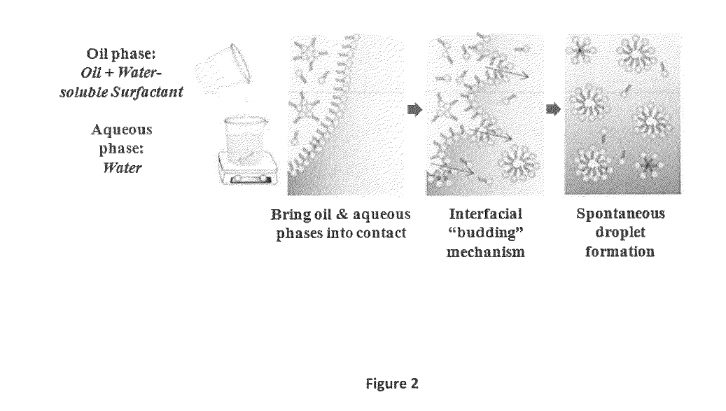

The aqueous phase composition must be carefully controlled to ensure small droplets are formed, that they are stable, and that they have appropriate functional properties. The aqueous phase composition can be controlled by varying the amount and/or types of water, surfactant, cosurfactant, cosolvent, buffers, and salts present, etc. These components can be added to the aqueous phase either before or after the organic phase is titrated into the aqueous phase to form the nanoemulsions (FIG. 2). A few selected examples are given below to highlight this point.

A. Nutraceutical ω-3 Nanoemulsions: Influence of Cosolvent

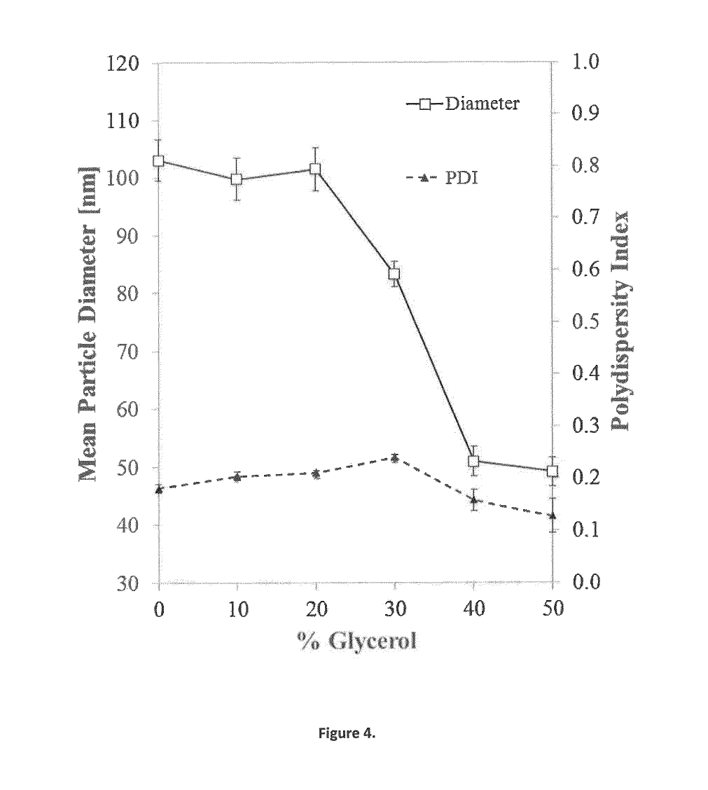

Droplet Formation: We produced ω-3 oil nanoemulsions with droplet diameter <50 nm by titrating an organic phase (fish oil, lemon oil, and a non-ionic food-grade surfactant (Tween 80)) into an aqueous phase (water and glycerol) (FIG. 4). The glycerol was used here as a water-soluble cosolvent. The mean diameter of the droplets decreased with increasing glycerol concentration, eventually reaching a minimum value. By carefully controlling the glycerol concentration in the aqueous phase it was possible to produce ultrafine droplets. For other cosolvents and oil types, we have found a minimum in the particle size versus cosolvent concentration profile at intermediate cosolvent concentrations. Consequently, the cosolvent type and concentration in the aqueous phase must be carefully optimized for the particular organic phase that is used.

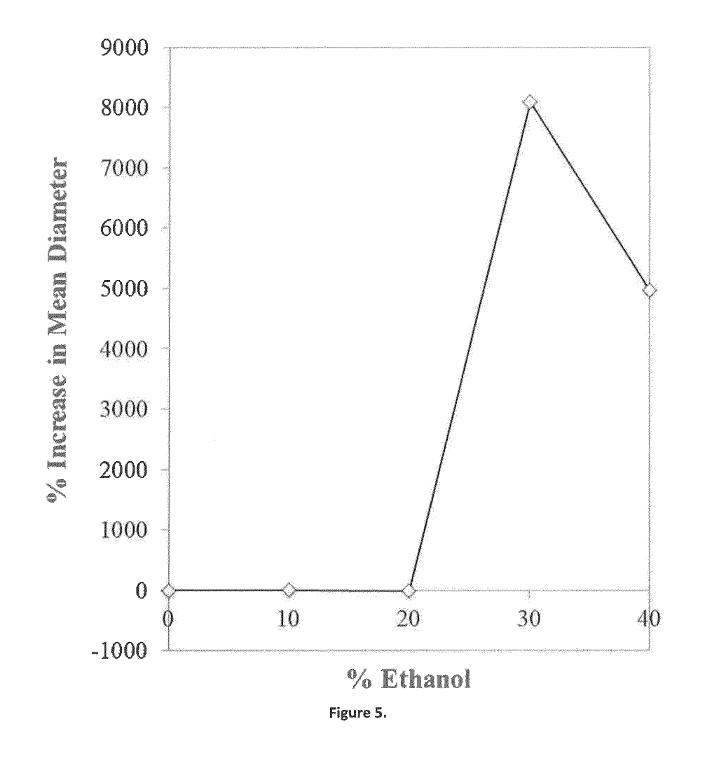

Droplet stability: The influence of cosolvent (ethanol) concentration on the increase in mean particle diameter after 24 hours storage of nanoemulsions produced by titrating an organic phase (fish oil, lemon oil, and Tween 80) into an aqueous phase (water and glycerol) using the spontaneous emulsification method is shown in FIG. 5 which shows the effect of ethanol concentration on increase in mean particle diameter after 24 hours storage of nanoemulsions produced by titrating an organic phase (fish oil, lemon oil, and Tween 80) into an aqueous phase (water and glycerol) using the spontaneous emulsification method. For this organic phase, the droplets were highly unstable to growth at high ethanol concentrations. The droplets were highly unstable to growth at high ethanol concentrations, which may have been due to droplet coalescence or Ostwald ripening. The influence of the cosolvent in the aqueous phase on droplet instability depends on the nature of the organic phase, and so must be optimized for each system.

The spontaneous emulsification method is very simple to carry out and can be used to encapsulate a wide range of lipophilic components by tailoring the cosolvent type and concentration to the oil type. e.g., oil soluble vitamins (Vitamin A, D and E), ω-3 oils (such as fish, algae, flax and krill oil), and conjugated linoleic acid (CLA).

2.2 Organic Phase Composition

The organic phase composition must be carefully controlled to ensure small droplets are formed, that they are stable, and that they have appropriate functional properties. The organic phase composition can be controlled by varying the amount and/or functional lipophilic component, carrier oil, surfactant, cosurfactant, and cosolvent present, etc. We show a few selected examples below to highlight this point.

Nutraceutical Nanoemulsions—Effect of Carrier Oil

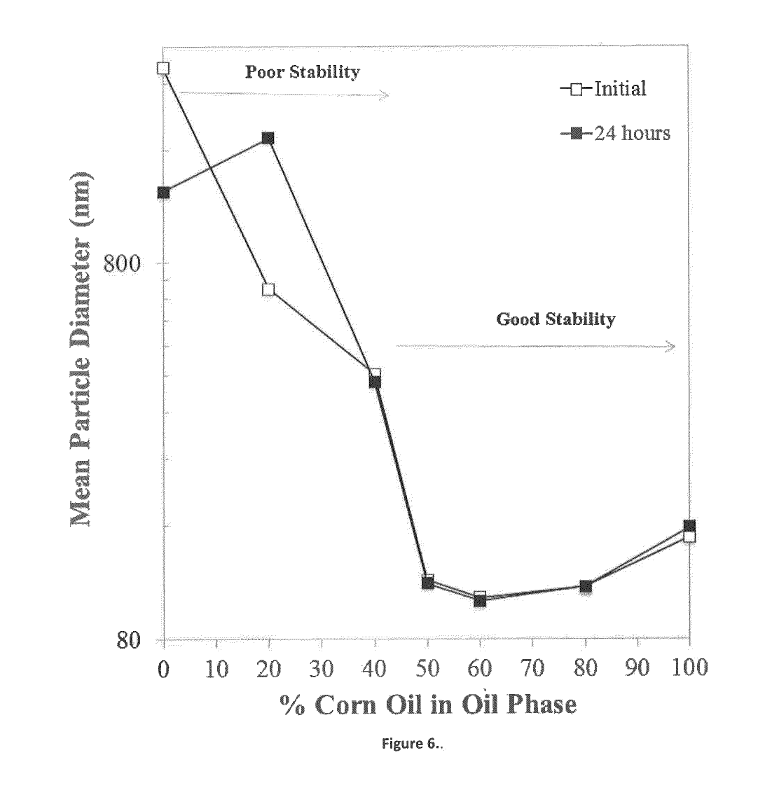

Droplet Formation: We produced ω-3 oil nanoemulsions with droplet diameter <90 nm by titrating an organic phase (fish oil and lemon oil) into an aqueous phase (FIG. 6 which shows the effect of lemon oil concentration on mean particle diameter of nanoemulsions produced using oil (fish oil and lemon oil), surfactant (Tween 80) and aqueous phase (ethanol and water) after 1 and 24 hours storage.). The mean diameter of the droplets initially decreased with increasing lemon oil concentration in the organic phase, then reached a minimum value, and then increased again. By carefully controlling the lemon concentration in the organic phase it was possible to produce ultrafine droplets. Consequently, the oil phase composition must be carefully controlled to form small oil droplets in the initial nanoemulsions.

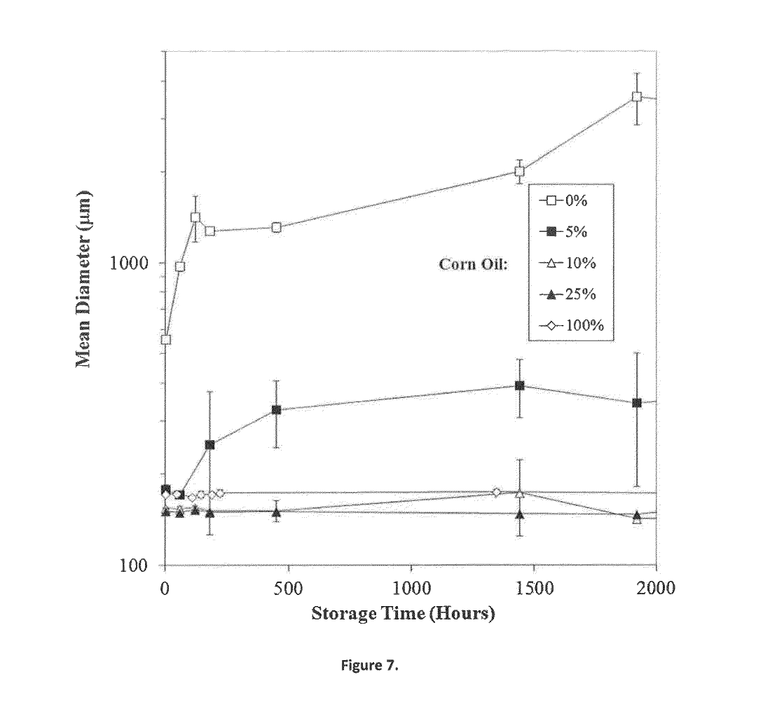

Droplet Stability: The nature of the carrier oil also influences the long-term stability of nanoemulsions after formation. In particular, the droplets may become unstable through a variety of mechanisms, including coalescence, Ostwald ripening, flocculation, partial coalescence and gravitational separation. The oil phase composition must therefore be carefully formulated to avoid this effect. This effect is clearly highlighted in FIG. 7, which shows the influence of corn oil on the stability of tributyrin nanoemulsions (produced using a high energy method). When the corn oil concentration is below a certain value (around 20%), rapid droplet growth occurs due to Ostwald ripening. Consequently, the oil phase must be optimized to prevent nanoemulsion instability after formation.

Potentially, a variety of different approaches can be used to improve the stability of nanoemulsions after preparation based on optimizing their organic phase composition: adding carrier oils; adding ripening inhibitors; adding surfactants/cosurfactants; adding cosolvents, etc.

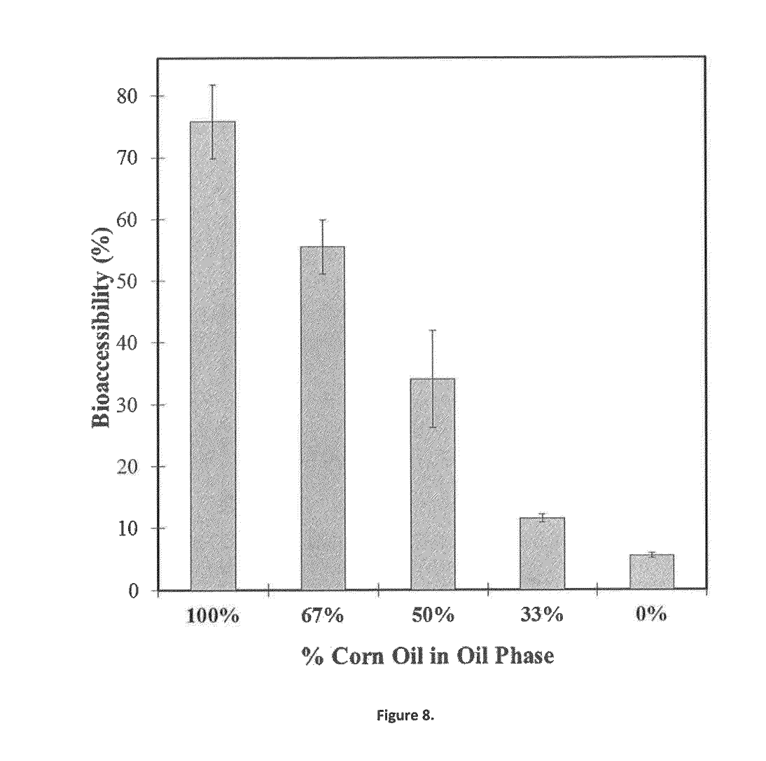

Nanoemulsion Functionality: The functional performance of nanoemulsions after they have been produced also depends strongly on oil type. Nanoemulsions may need to have many different kinds of functional performance, e.g., appearance, texture, flavor, antimicrobial activity, bioactivity, etc. As an example, we show the influence of oil phase composition on the bioaccessibility of an encapsulated bioactive component (β-carotene). As the ratio of digestible oil (corn oil) to indigestible oil (lemon oil) decreases, the bioaccessibility decreases because less mixed micelles are formed to solubilize the bioactive components after lipid digestion (FIG. 8 which shows the effect of oil phase composition (% corn oil in oil phase) on the bioaccessibility of encapsulated β-carotene in nanoemulsions produced using oil (lemon oil and corn oil), surfactant (sucrose monopalmitate) and aqueous phase (water). The bioaccessibility decreases as the concentration of corn oil decreases.). This study highlights the importance of controlling the organic phase composition to ensure good functional performance of nanoemulsions after they have been prepared.

B. Antimicrobial Essential Oil Nanoemulsions—Effect of Carrier Oil

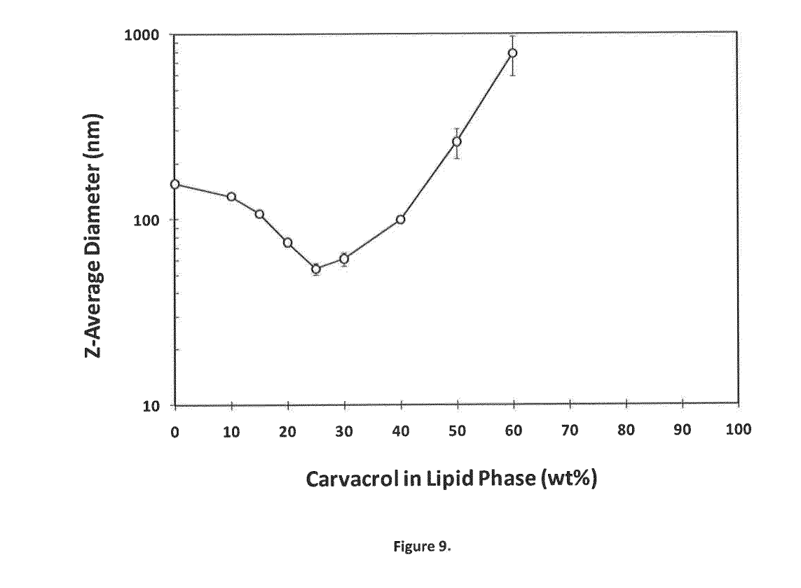

Droplet Formation: Oil phase composition (carvacrol to medium chain triglyceride (MCT) ratio) had a major effect on nanoemulsion formation (FIG. 5), with the smallest droplets being formed at 2.5 wt % carvacrol and 7.5 wt % MCT (r≈26 nm). Carvacrol alone was unable to form stable nanoemulsions by spontaneous emulsification: the emulsions produced were extremely unstable and rapidly separated, which was attributed to Ostwald ripening and/or coalescence (FIG. 9 that shows the effect of oil phase composition (% wt of carvacrol in oil phase) on mean particle diameter of emulsions and nanoemulsions produced by spontaneous emulsification. Emulsions and nanoemulsions were prepared using 10 wt % oil (Carvacrol+MCT), 10 wt % surfactant (TWEEN® 80) and 80 wt % water (pH 3.5 citrate buffer solution) at a stirring speed of 500 rpm at ambient temperature (≈25° C.). Only if carvacrol was mixed with a certain amount of carrier oil (MCT), could stable emulsions be formed. For example, when carvacrol levels in the lipid phase were 60 or 80 wt %, the systems formed were highly unstable and phase separation started quickly. When the carvacrol level was further decreased to 50 wt % in the oil phase, temporarily stable emulsions could be formed with relatively large droplets diameters (d≈800 nm) (FIG. 9), but these emulsions were unstable after a few days storage and oiling off could be observed on the top. At lower levels of the essential oil in the oil phase (≦40 wt %) it was possible to form nanoemulsions (i.e., d<200 nm). It should be noted the selection of carrier oil was also critical: oils with long chain fatty acids (like corn oil, canola oil) were unable to form stable carvacrol nanoemulsions at any usage levels.

Droplet Stability: The organic phase composition may also influence the long-term stability of the nanoemulsions. For, example it may be necessary to optimize the system to produce small droplets, but that also have good stability, which often work in opposite directions. For example, a carrier oil may act as a ripening inhibitor that prevents droplet growth after nanoemulsion formation by inhibiting Ostwald ripening. However, if too much is added to the oil phase then the system will not be able to form a nanoemulsion in the first place.

Nanoemulsion Functionality: The oil phase composition may also influence the functional performance of the nanoemulsions. For, example it may be necessary to optimize the system to produce stable small droplets, but that also have good functional performance (such as antimicrobial activity, bioavailability, or chemical stability), which often work in opposite directions. As an example of this effect we use the illustration of the influence of oil phase composition on the antimicrobial efficacy of essential oil nanoemulsions.

We determined the antimicrobial effects of different essential oil (carvacrol) nanoemulsions by measuring their minimum inhibitory concentration (MIC) in a nutrient MEB medium (pH 3.5), against four acid resistant yeast strains: Zygosaccharomyces bailii (ZB), Saccharomyces cerevisiae (SC), Brettanomyces bruxellensis (BB), and Brettanomyces naardenensis (BN). The MICs were calculated as carvacrol mass concentration in the final broth.

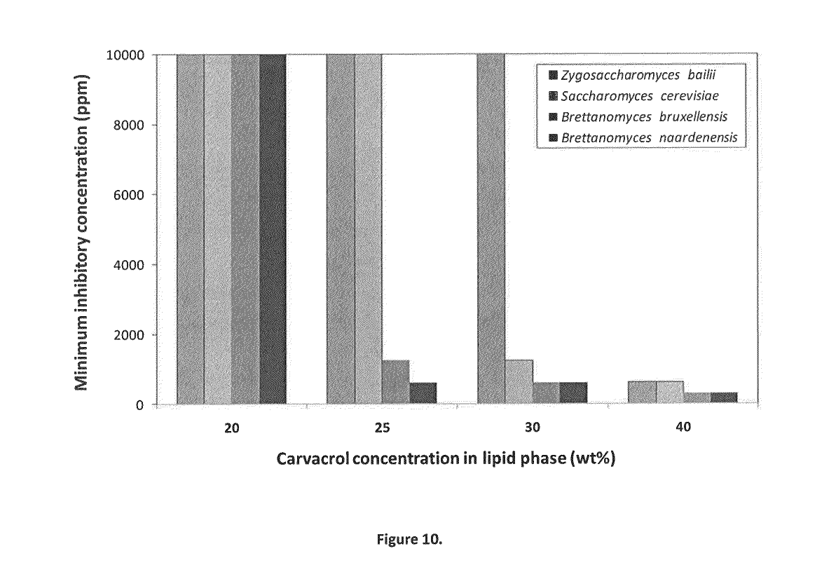

The antimicrobial efficacy of the nanoemulsions was highly related to the relative amounts of carvacrol in the oil phase: the higher carvacrol concentration in the oil phase, the better antimicrobial efficacy (lower MIC values, i.e., less amounts of carvacrol are needed to completely inhibit yeast growths) (FIG. 10). For example, when carvacrol concentration was 20 wt % or less in the oil phase, even 1 wt % (10,000 ppm) of carvacrol could not inhibit growth by either of the four test yeasts.

When carvacrol concentration was 40 wt % in the lipid phase (i.e., 4 wt % Carvacrol+6 wt % MCT in the initial emulsion system), the carvacrol nanoemulsion had most strong antimicrobial efficacy, and the growths of all four test yeast strains could be completely inhibited by only 625 ppm of carvacrol. If the carvacrol concentration was lower, the nanoemulsions were not very effective at inhibiting microbial growth. However, if not enough MCT was added, the systems were physically unstable. Hence it was necessary to carefully control oil phase composition to form small droplets, ensure their stability, and ensure their functional performance.

Antimicrobial Essential Oil Nanoemulsions—Effect of Surfactant

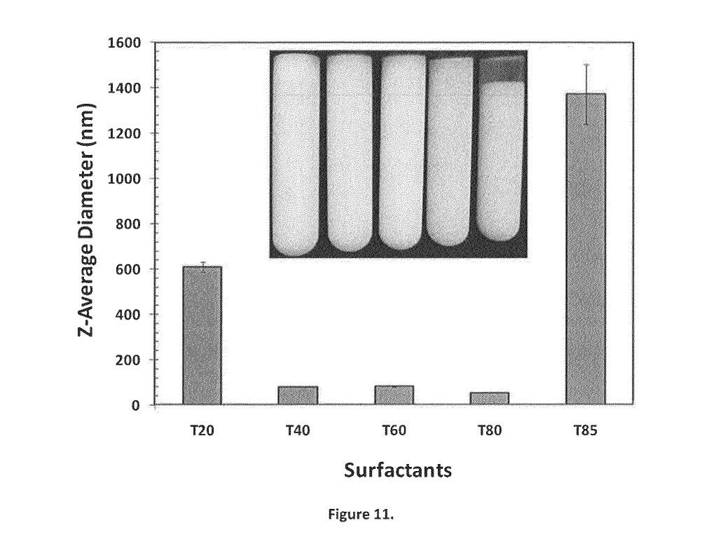

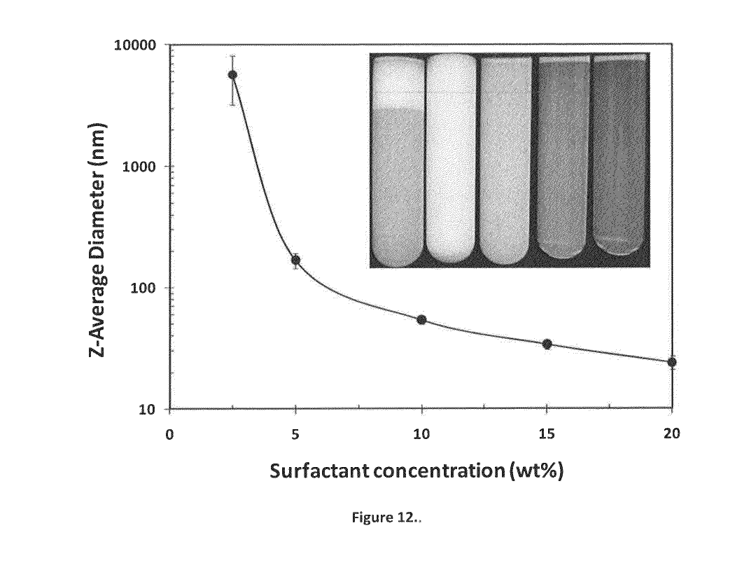

The type and concentration of the surfactant present within the organic phase also has a major impact on the size, stability, and functional performance of the nanoemulsions. The effect of surfactant type on particle size and stability of nanoemulsions is shown in FIG. 11: TWEEN® 80 giving the smallest droplets from a group of food-grade non-ionic surfactants (TWEEN® 20, 40, 60, 80, and 85). The surfactant concentration also affected the size of the nanoemulsions (FIG. 12), with smaller droplets being formed at higher surfactant concentrations. In other cases, we have found that a minimum occurs in the droplet size at a particular surfactant concentration, which highlights the need to optimize this parameter for the particular system in question.

C. Fabrication of Orange Oil Nanoemulsions

Clear orange oil nanoemulsions have been fabricated by a low energy method of spontaneous emulsification. Oil phase composition (mass ration of orange oil to MCT), surfactant levels, surfactant types, all have appreciate effect on the formation of orange oil nanoemulsions. Results, using the present teachings, showed that clear nanoemulsions can be formed under certain system conditions (20% Tween40, Tween60 or Tween80; 4%-6% orange oil in a total of 10 wt % oil phase with the helper oil being MCT).

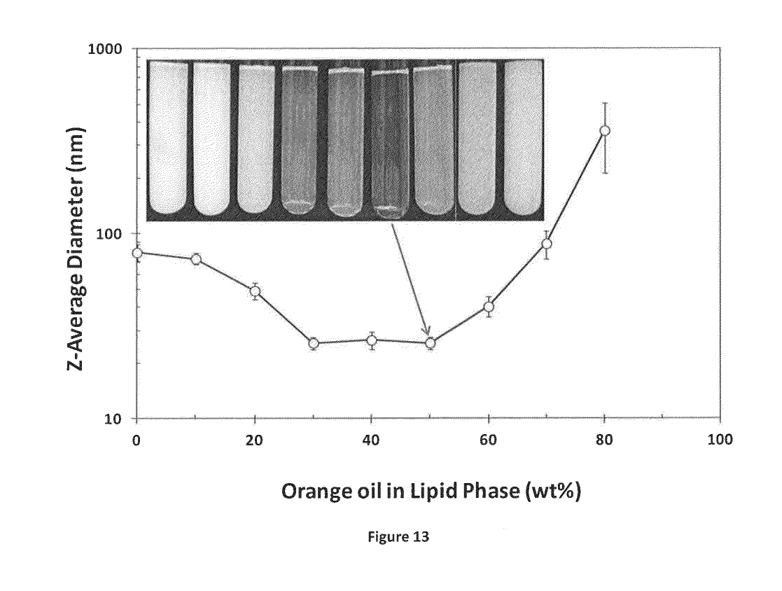

FIG. 13 shows the Effect of oil phase composition (% wt of orange oil in oil phase) on mean particle diameter of emulsions and nanoemulsions produced by spontaneous emulsification. Emulsions and nanoemulsions were prepared using 10 wt % oil (orange oil+MCT), 10 wt % surfactant (TWEEN® 80) and 80 wt % water (pH 3.5 citrate buffer solution) at a stirring speed of 500 rpm at ambient temperature (≈25° C.). From left to right, the photograph shows orange oil concentrations in the lipid phase of 0, 10, 15, 20, 25, 30, 40, 50, 60, 80, 100 wt %, respectively.

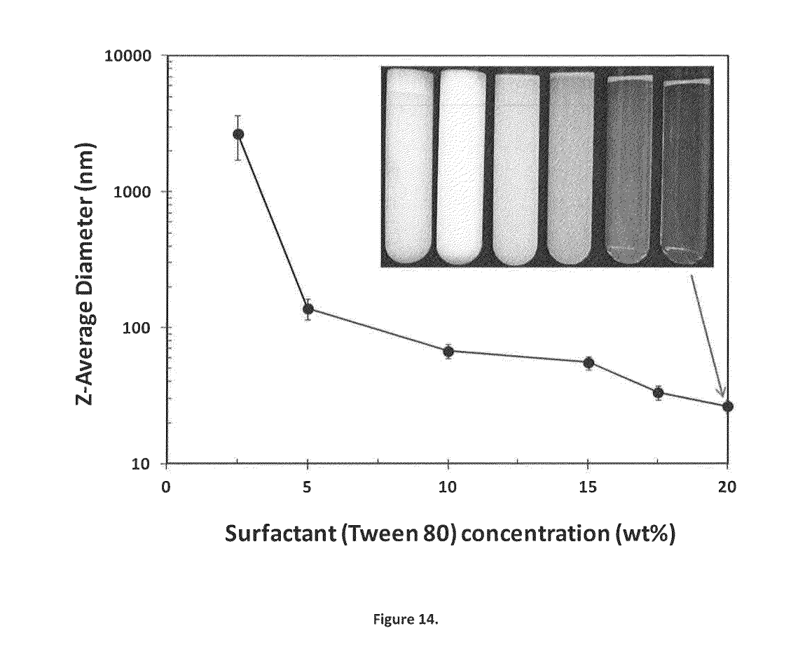

FIG. 14 shows the Effect of surfactant concentration on mean particle diameter and physical stability of emulsions produced by spontaneous emulsification. Surfactant (Tween 80) concentration in emulsion varied from 2.5 to 20 wt % as indicated. Oil phase=4% orange oil+6% MCT.

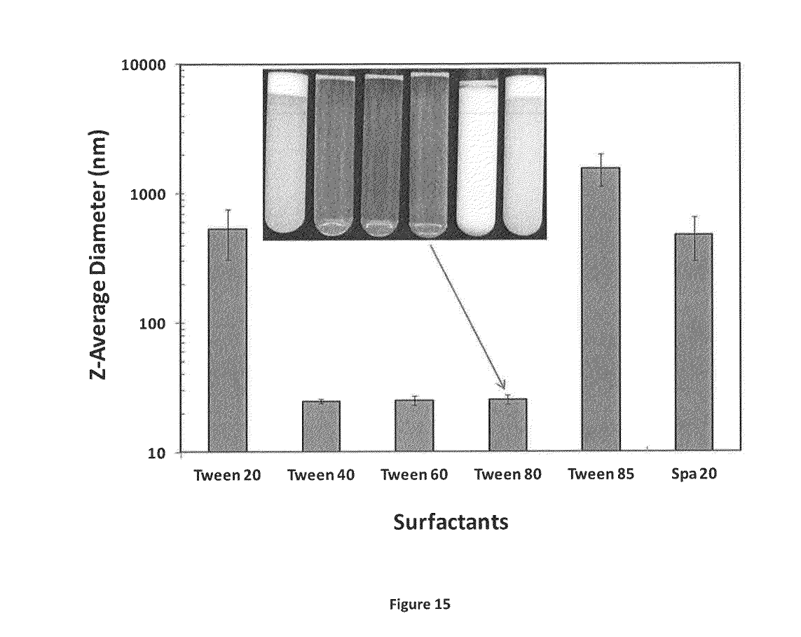

FIG. 15 shows the Effect of surfactant type on mean particle diameter and physical stability of emulsions produced by spontaneous emulsification. Oil phase=4% orange oil+6% MCT. Surfactant was 20 wt % in system.

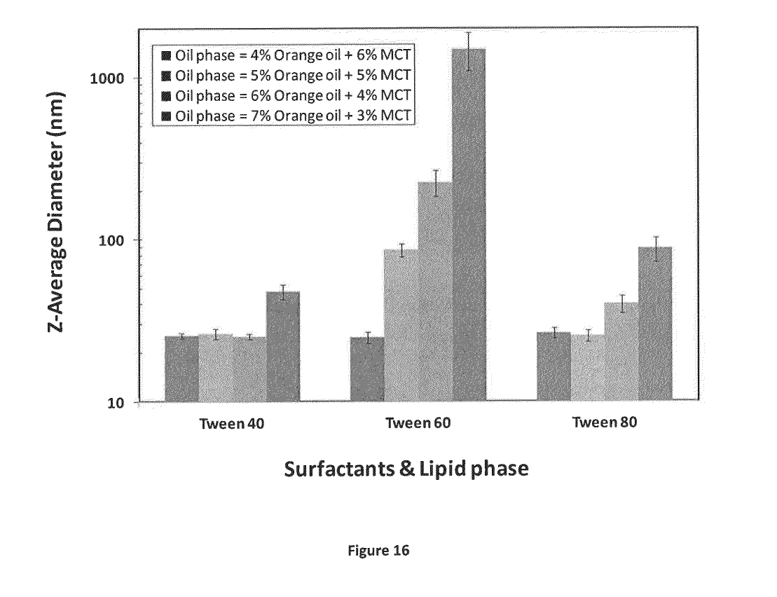

FIG. 16 shows the Effect of surfactant type and oil phase composition on mean particle diameter of emulsions produced by spontaneous emulsification. Oil phase composition (varying ration of orange oil+MCT, total oil=10 wt %) was indicated as in the legend. Surfactant was 20 wt % in system. The results indicate that Tween 40 can load most amount of orange oil (6%) producing transparent nanoemulsions with very small particle sizes (d≈25 nm), while Tween 60 can load least amount of orange oil (4%), and Tween 80 in the middle (5% orange oil). Other types of surfactants cannot produce transparent orange oil nanoemulsions, no matter of the oil phase.

Further details of the exemplary embodiment entitled Nutraceutical ω-3 Nanoemulsions: Influence of Cosolvent and further details of the exemplary embodiments entitled Antimicrobial Essential Oil Nanoemulsions are presented in Appendix I and Appendix II of U.S. Provisional Application No. 61/865,778, entitled METHODS FOR PRODUCING OPTIMAL STABLE NANOEMULSIONS AND FORMULATIONS OBTAINED THEREFROM, filed on Aug. 14, 2013, the entire contents of which are incorporated herein by reference for all purposes.

For the purposes of describing and defining the present teachings, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Although the invention has been described with respect to various embodiments, it should be realized this invention is also capable of a wide variety of further and other embodiments within the spirit and scope of the appended claims.

1. A method for producing stable nanoemulsions having a desired droplet size and functional properties tailored for use in a predetermined application, the method comprising:
selecting an aqueous phase, the aqueous phase comprising at least one ingredient from water, surfactant, co-surfactant and co-solvent, type and concentration of the aqueous phase being selected such that a stable nanoemulsion having a desired droplet size and functional properties tailored for use in the predetermined application is obtained;
selecting an organic phase comprising at least two ingredients from lipophilic component, oil, surfactant, co-surfactant and cosolvent, type and concentration of the organic phase being selected such that a stable nanoemulsions having a desired droplet size and functional properties tailored for use in the predetermined application is obtained; and
mixing the organic phase with the aqueous phase; a nanoemulsion being formed when the organic phase is mixed with the aqueous phase.

2. The method of claim 1 wherein selecting the organic phase comprises varying type and concentration of the at least two ingredients selected from lipophilic component, oil, surfactant, co-surfactant and cosolvent.

3. The method of claim 1 wherein selecting the aqueous phase comprises varying type and concentration of the at least one ingredient selected from water, surfactant, co-surfactant and co-solvent.

4. The method of claim 3 wherein selecting the aqueous phase also comprises varying type and concentration of ingredients selected from buffers and salts.

5. The method of claim 4 wherein selecting the aqueous phase also comprises varying pH.

6. The method of claim 2 wherein selecting the aqueous phase comprises varying type and concentration of the at least one ingredient selected from water, surfactant, co-surfactant and co-solvent; and wherein type and concentration of ingredients of the aqueous phase and type and concentration of ingredients of the organic phase are varied substantially simultaneously.

7. The method of claim 1 wherein, after producing a stable nanoemulsion having a desired droplet size and functional properties tailored for use in a predetermined application, type and concentration of at least two organic phase ingredients selected from lipophilic component, oil, surfactant, co-surfactant and cosolvent are varied in order to ensure stability during manufacturing, storage, handling and utilization.

8. The method of claim 1 wherein, after producing a stable nanoemulsion having a desired droplet size and functional properties tailored for use in a predetermined application, type and concentration of at least two aqueous phase ingredients selected from water, surfactant, co-surfactant and co-solvent are varied in order to ensure stability during manufacturing, storage, handling and utilization.

9. The method of claim 8 wherein type and concentration of ingredients selected from buffers and salts are varied in order to ensure stability during manufacturing, storage, handling and utilization.

10. The method of claim 9 further comprising varying pH.

11. The method of claim 10 wherein type and concentration of at least two organic phase ingredients selected from lipophilic component, oil, surfactant, co-surfactant and cosolvent are varied in order to ensure stability during manufacturing, storage, handling and utilization.

12. The method of claim 1 wherein the organic phase comprises two or more oils; and wherein selecting the organic phase comprises varying a mass ratio of each of the two or more oils.

13. The method of claim 1 wherein the aqueous phase comprises two or more polar components; and wherein selecting the aqueous phase comprises varying a mass ratio of each of the two or more polar components.

14. The method of claim 1 wherein the organic phase comprises two or more surfactants; and wherein selecting the organic phase comprises varying a mass ratio of each of the two or more surfactants.

15. The method of claim 1 wherein the organic phase comprises surfactant and oil; and wherein selecting the organic phase such that a stable nanoemulsions having a desired droplet size and functional properties tailored for use in the predetermined application is obtained comprises selecting a ratio of surfactant to oil.

16. The method of claim 15 wherein selecting a ratio of surfactant to oil comprises varying a mass ratio of the surfactant and a mass ratio of the oil to obtain a surfactant to oil ratio such that a stable nanoemulsions having a desired droplet size and functional properties tailored for use in the predetermined application is obtained.

17. The method of claim 1 wherein the organic phase comprises surfactant and oil; wherein the aqueous phase comprises water; and wherein a concentration of surfactant and oil in a surfactant, oil and water mixture is selected such that a stable nanoemulsions having a desired droplet size and functional properties tailored for use in the predetermined application is obtained.

18. The method of claim 17 wherein the concentration of surfactant and oil in a surfactant, oil and water mixture is selected by varying a mass ratio of the surfactant, a mass ratio of the oil and a mass ratio of the water.

19. The method of claim 1 further comprising diluting the nanoemulsion by a predetermined dilution factor, the dilution factor selected to achieve long-term stability.

20. The method of claim 19 wherein the nanoemulsion is diluted with water.

21. The method of claim 19 wherein the nanoemulsion is diluted with an aqueous solution comprising stabilizing components.

22. The method of claim 21 wherein the stabilizing components is selected from at least one of surfactants, minerals, buffers, proteins, polysaccharides and antioxidants.

23. A formulation comprising:
an aqueous phase, the aqueous phase comprising:
at least one ingredient from water, surfactant, co-surfactant and co-solvent, type and concentration of the aqueous phase being selected such that a stable nanoemulsion having a desired droplet size and functional properties tailored for use in a predetermined application is obtained;
an organic phase comprising:
at least two ingredients from a lipophilic component, oil, surfactant, co-surfactant and cosolvent, type and concentration of the organic phase being selected such that a stable nanoemulsion having a desired droplet size and functional properties tailored for use in the predetermined application is obtained;
a nanoemulsion being formed when the organic phase is mixed with the aqueous phase.

24. The formulation of claim 23 wherein the aqueous phase also comprises buffers and salts.

25. The formulation of claim 23 wherein the organic phase comprises an oil phase comprising at least two oils blended together at different mass rations; the at least two oils and the different mass rations being selected such that a stable nanoemulsion having a desired droplet size and functional properties tailored for specific use in a predetermined application is obtained.

26. The formulation of claim 23 wherein the aqueous phase comprises at least two polar components blended together a different mass rations; the at least two for components and the different mass rations being selected such that a stable nanoemulsion having a desired droplet size and functional properties tailored for specific use in a predetermined application is obtained.

27. The formulation of claim 25 wherein the organic phase also comprises at least two surfactants blended at other different mass rations; the at least two surfactants and the other different mass rations being selected such that a stable nanoemulsion having a desired droplet size and functional properties tailored for specific use in a predetermined application is obtained.

28. The formulation of claim 23 wherein the organic phase comprises surfactant and oil; and wherein a ratio of surfactant to oil is selected such that a stable nanoemulsion having a desired droplet size and functional properties tailored for use in the predetermined application is obtained.

29. The formulation of claim 23 wherein the organic phase comprises surfactant and oil; wherein the aqueous phase comprises water; and wherein a concentration of surfactant and oil in a surfactant, oil and water mixture is selected such that a stable nanoemulsion having a desired droplet size and functional properties tailored for use in the predetermined application is obtained.

30. The formulation of claim 23 further comprising another aqueous solution comprising stabilizing components.

31. The formulation of claim 30 wherein the stabilizing components are selected from at least one of surfactants, minerals, buffers, proteins, polysaccharides and antioxidants.
